(19) United States
(12) Patent Application Publication
(10) Pub. No.: US 2016/0059353 A1
(43) Pub. Date: Mar. 3, 2016

(54) SURFACE STRUCTURING OF METALS

(71) Applicant: NEWSOUTH INNOVATIONS PTY LIMITED, New South Wales (AU)

(72) Inventor: Christopher William Douglas Dodds, New South Wales (AU)

(21) Appl. No.: 14/888,631

(22) PCT Filed: May 5, 2014
(86) PCT No.: PCT/AU2014/000495

§ 371 Date: Nov. 2, 2015

(30) Foreign Application Priority Data
May 3, 2013 (AU) 2013901583

(51) Int. Cl. B23K 26/352; B23K 26/0622; A61N 1/05; B23K 26/00

(52) U.S. Cl. CPC B23K 26/352; B23K 26/0078; B23K 26/0624; A61N 1/0543; A61N 1/0541; A61N 1/0551; A61N 1/0534

(57) ABSTRACT

A method of treating a metallic surface comprising exposing the surface to laser pulses at an energy density below the threshold for ablation of bulk material from the metallic surface; maintaining the exposure until a multiplicity of pores form in the surface.

### TECHNICAL FIELD

This invention relates to surface structuring of metals and particularly to methods for increasing the electrochemical surface area of metallic surfaces.

### BACKGROUND

Surfaces having a high electrochemical surface area are particularly desirable in electrode applications and in any applications which require a metallic surface having an increased surface area. Typical methods of providing a surface having an increased electrochemical surface area include depositing a coating using electrochemical means to create a high electrochemical surface area. Other known methods include the use of harsh chemicals or other contact to etch or create surface roughness elements.

### SUMMARY OF THE DISCLOSURE

Disclosed is a method of treating a metallic surface comprising exposing the surface to laser pulses at an energy density below the threshold for ablation of bulk material from the metallic surface; maintaining the exposure until a multiplicity of pores form in the surface.

Further disclosed is a method of enhancing the electrochemical surface area of an electrode comprising treating the surface of the electrode by applying laser pulses at an energy density below the threshold for ablation of bulk material for the surface of the electrode and repeating the exposure until a porous surface is formed on the electrode.

Further disclosed is a method of forming an electrode for use within a medical device, the method comprising: treating a surface of a platinum electrode by exposing the surface to laser pulses at an energy density below the threshold for ablation of bulk platinum; maintaining the exposure until a multiplicity of pores form in the surface.

The method provides an efficient technique for providing a surface having a high electrochemical surface area without contact, harsh chemicals or deposition. This is of specific benefit in electrode manufacture particularly in applications such as medical applications including retinal implants, visual prostheses, cochlea implants, peripheral nerve implants, deep brain stimulators and other applications which require electrodes.

### BRIEF DESCRIPTION OF THE FIGURES

Embodiments will now be described by way of example only, with reference to the accompanying drawings in which

FIG. 1 shows a metallic surface having undergone the method of treatment of one embodiment of the disclosure;

FIG. 2 shows a laser scanning patterns for structuring Pt with A. square pattern and B. triangular pattern;

FIG. 3 shows light and SEM images showing the representative surfaces for each laser pattern on Pt microelectrodes;

FIG. 4 is a graphical representation of the surface index of treated electrodes;

FIG. 5 is a graphical representation of the charge storage capacity or treated electrodes;

FIG. 6 is a graphical representation of impedance behavior of treated electrodes;

FIG. 7 is a graphical representation of the charge injection limit of treated electrodes;

FIG. 8 is a graphical representation of charge injection limits for treated electrodes;

FIG. 9 is a graphical representation of charge injection limits with respect to visual percept thresholds;

FIG. 10 is a graphical representation of voltage on treated electrodes;

FIG. 11 is a graphical representation of voltage on further treated electrodes;

FIG. 12 shows gas bubbles present on electrode sites of an array.

### DETAILED DESCRIPTION OF EMBODIMENTS

The term “metallic surface” is used in this disclosure to refer to any type of metallic surface suitable for surface treatment for increased electrochemical surface area. The detailed description refers in particular to metallic surfaces of electrodes. Electrode efficiencies are proportional to the electrochemical surface area of the electrode rendering increasing the electrochemical surface area significant. However while the metallic surfaces of electrodes are a key application of the technology, it will be clear to a person with knowledge of metallic surfaces and surface areas that the disclosure may be directed toward alternative metallic surfaces warranting increased electrochemical surface area.

In the following detailed description, reference is made to accompanying drawings which form a part of the detailed description.

In some disclosed forms, disclosed is a method of treating a metallic surface comprising exposing the surface to laser pulses at an energy density below the threshold for ablation of bulk material for the metallic surface and maintaining the exposure until a multiplicity of pores form in the surface.

In some forms the metallic surface is the surface of a platinum electrode.

In some forms the exposure is maintained through a rippled surface effect until a porous surface structure is produced.

In some forms the step of exposing the surface to laser pulses is performed greater than 1000 times. In some forms the surface is exposed to greater than 20,000 laser pulses. In some forms the surface is exposed to greater than 25,000 laser pulses.

Further disclosed is a method of enhancing the electrochemical surface area of an electrode comprising treating the surface of the electrode by applying laser pulses at an energy density below the threshold for ablation of bulk material for the surface of the electrode and repeating the exposure until a porous surface is formed on the electrode.

In some forms the exposure is maintained through a rippled surface effect until a porous surface structure is produced on the electrode.

Further disclosed is a method of forming an electrode for use within a medical device, the method comprising: treating a surface of a platinum electrode by exposing the surface to laser pulses at an energy density below the threshold for ablation of bulk material for platinum; maintaining the exposure until a multiplicity of pores form in the surface.

In some forms the medical device is a visual prosthesis. In some forms the medical device is a retinal implant. In some forms the medical device is a cochlea implant. In some forms the medical device is a peripheral nerve implant. In some forms the medical device is a deep brain stimulator.

Generally the application discloses a method of treating a metallic surface. The method results in an increased electrochemical surface area by forming surface pores, micropitting or alternative roughness elements upon or within the metallic surface. After treatment the surface includes raised or lowered elements and spaces therebetween which act to greatly enhance the surface area of the surface.

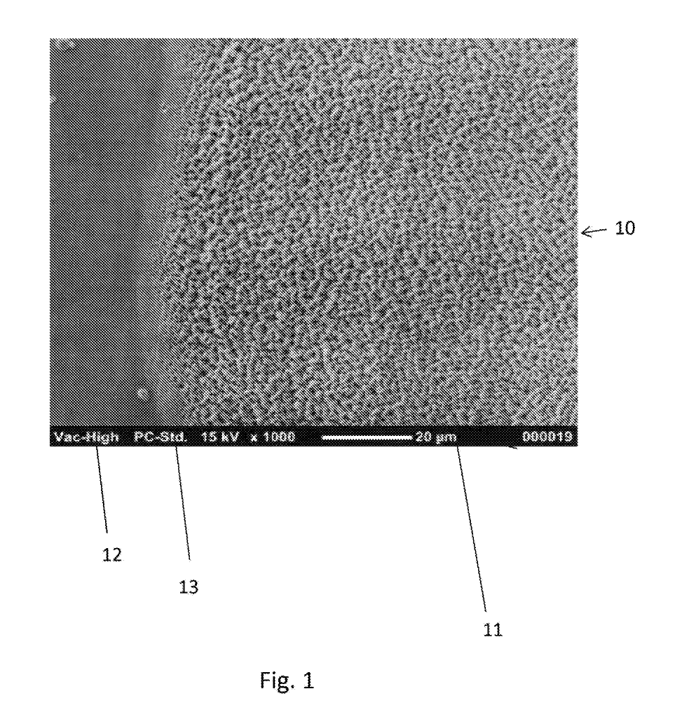

In one embodiment the method comprises illuminating a metallic surface 10 with laser pulses, more particularly ultra-short laser pulses. The laser pulses to which the metallic surface is exposed have an energy density below the threshold for ablation of bulk material for the metallic surface. The metallic surface is exposed to the laser pulses repeatedly until a porous structure 11 having a high surface area is formed at the metallic surface. FIG. 1 further shows an untreated region 12 of the surface and a border region 13 which is intermediate the untreated region 12 and the porous structure 11.

The method results in surface plasmon interference effects at the metallic surface. Continued exposure to the laser pulses results in areas of energy density above the energy level of ablation which results in a rippled effect that is compounded to form micropores within a porous structure.

In some forms and examples, the step of exposing the surface 10 to laser pulses is performed utilising a scanner head to deflect the beam path of the laser pulses across the metallic surface of, for example, an electrode. In order to expose an area larger than the laser beam width, the pulses proceeded in a series of parallel lines. The angle of the lines was changed and then the laser pulses illuminated the surface again. The steps are performed a multiplicity of times. In one form the steps are performed greater than 1000 times, such that each point on the surface is exposed to the laser pulses in the order of 25000 times.

Through the process of exposing the surface to laser pulses results in an initially rippled or striated surface pattern. However repeatedly applying many laser pulses to the metallic surface results in a dramatically changed surface morphology, creating a surface having micropores or pits therein.

The increased surface area is highly desirable in the production of electrodes, particularly those for applications in which miniaturisation is optimal for health or other purposes. The method allows for adjustment of the surface roughness. This allows for adjustment of the parameters to create a surface of low to moderate roughness with large pores. While the electrochemical surface area of the metallic surface is limited with a low or moderate surface roughness the decrease in roughness provides benefits in not becoming congested with larger molecules or proteins and maintains a mechanically strong electrode structure. The method can therefore be utilised to provide electrodes having a high electrochemical surface area without creating a surface that is too fine, leading to pore clogging on the surface or surface fragility.

In one application, electrodes formed by the disclosed method are utilized within a retinal implant for enabling patients to perceive vision through stimulating phosphenes. The more electrodes an implant contains, the more phosphenes are capable of being generated and the more detail a patient may be able to see.

In alternative embodiments the nature, roughness, pore size can be altered through controlling the energy and positioning of laser pulses.

The method allows for production of a metallic surface with a higher electrochemical surface area while avoiding or reducing the need to expose them to harsh chemicals or deposition techniques. In some forms and in some manufacturers lasers are presently available for creation of high density electrode arrays for use in, for example, neuroprosthetics.

In one form, the laser used has a pulse duration of 12 ps, a wavelength of 532 nm and a beam width of 10 um. The laser is first directed across a test surface of the target material at varying intensities, to determine the maximum power which does not result in ablation of the bulk material. The target is then processed by deflecting the laser beam at this power across the surface, in a series of parallel lines spaced 8 um apart, allowing for some overlap to occur. In successive passes, the direction of the parallel lines is rotated by 60 and 120 degrees. In one form the scanning process is repeated 1000 times.

### EXAMPLES

### Introduction

Laser surface modification of platinum (Pt) electrodes was investigated for use in neuroprosthetics. Surface modification was applied to increase the surface area of the electrode and improve its ability to transfer charge within safe electrochemical stimulation limits. Electrode arrays were laser micromachined to produce Pt electrodes with smooth surfaces, which were then modified with four laser patterning techniques to produce surface structures which were Nd:YAG patterned, square profile, triangular profile and roughened on the micron scale through structured laser interference patterning

The voltage produced across an electrode for a particular charge density is inversely proportional to the surface area. As a result, larger electrodes are able to inject more charge before exceeding the electrochemically safe limits. However, larger electrodes consume greater space and as a result, limit the spatial selectivity or resolution of a device produced from such electrodes. Assuming close apposition with target neural tissue, a greater number of electrodes enables the delivery of a higher resolution signal, which is expected to translate to an improvement in sound perception for cochlear implant users and increased visual acuity for visual prosthesis recipients. Due to the space limitations within organs such as the cochlea and eye, increasing the number of electrodes without reducing the size of the electrodes is not possible. However, reducing the size of an electrode will significantly reduce the charge which can be delivered while still preventing harmful chemical reactions. An alternative way to produce smaller electrodes or alternately increase the safety margin of existing electrodes, is to increase the electrode surface area.

Increases in the electrode charge transfer area can be achieved through surface modification or coatings. Physical methods involve using a laser to etch or melt the surface of the electrode. Green et al. found that the surface area of an electrode was increased by 2.6 times using an Nd:YAG laser to roughen the surface of the electrode. The safe charge injection limit was increased by 3.7 times at 0.5 ms phase length. Using similar laser melt methods Schuettler found a 4.5 times increase in the surface of the electrode and a decrease in polarisation voltage by 37%. However, Green et al. also found that the surface achieved by melt processing imparted from the relatively long laser pulse (1-100 μs) required for Nd:YAG roughening increased surface bound oxides of Pt, preventing the full electrode area from being utilised to transduce charge.

### Fabrication of Electrode Arrays

Electrodes were fabricated from laser micromachined Pt foils insulated in poly(dimethyl siloxane) (PDMS). The PDMS was spun onto a supporting microscope slide with a polyimide tape release layer. Following curing of the lower PDMS layer a Pt foil was laminated onto the slide and the Pt tracks and electrode sites were structured. This CAD guided laser micromachining process was designed to achieve electrode sites having a nominal diameter of 380 μm. An overlying PDMS layer was spun onto the supporting slide and cured to insulate the whole structure.

Opening of the PDMS above the electrode sites was performed using an excimer laser (Atlex 300 SI, ATL, Germany) with an ArF gas mixture to produce a 193 nm beam. The voltage was set to 15 kV and the frequency to 100 Hz. The beam was passed through a mask to irradiate a 50 μm circle on the target area of PDMS. The irradiated circle was directed in a circular path 335 μm in diameter at 50 μm/s to ablate the outline of the electrode site. This was repeated until the ablation depth was sufficient to expose the underlying Pt foil (typically 3-4 passes). The central disc of silicone was then removed manually.

Nd:YAG laser (GenesisMarker, ACI, Germany) roughening was performed using a 1064 nm beam with a spot size of 25 μm. Laser power was set to 12%, and a pulse repetition frequency of 700 Hz and pulse duration of 1 μs. The beam was deflected at a speed of 5 mm/s across the electrode surface in a series of parallel lines spaced 30 μm apart.

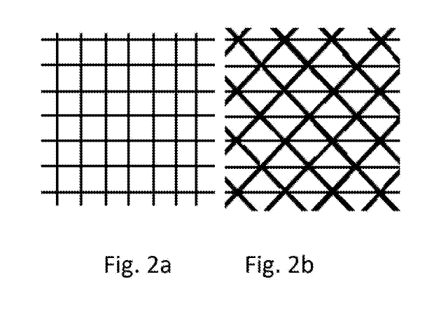

Three roughening patterns were produced using a picosecond, mode-locked laser (Duetto, Time-BandWidth, Switzerland). The laser was fitted with a second harmonic generator (SHG) to produce a 532 nm beam with a spot size of 11 μm. For all patterns, a pulse repetition frequency of 340 kHz was used. A square hatch pattern, depicted in FIG. 2a, was produced by scanning the beam across the electrode surface at 250 mm/s in a series of parallel lines spaced 20 μm apart, then repeating the process with a second series of lines arranged at 90° to the first. A triangular hatch pattern, FIG. 2b, was produced in a similar method, but using three series of lines spaced 60° apart. For both of these patterns, laser power was set at 80% of maximum. For the square pattern, eight repetitions were required to achieve the desired depth. For the triangular pattern, five repetitions were needed. The third structured laser interference pattern (SLIP) was produced by dropping the power to 32% but increasing the number of repetitions to 1000. The scanning pattern was similar to the triangular pattern outlined above, but with the line spacing reduced to 8 μm to produce interference between adjacent tracks.

Electrode arrays were visualised under scanning electron microscopy (SEM) and optical profilometry to confirm the surface patterning and quantify the surface roughness. Samples were visualised using SEM at 10 kV and 270× magnification (JEOL Neoscope). A GTK1-M Contour light interference profilometer (Bruker, USA) with a 20× magnification and Vision 64 software was used to generate a surface profile. The data was masked to eliminate the insulation material and enable calculation of the electrode site absolute surface area.

### Baseline Electrical Characterisation

A three-electrode cell was used to perform cyclic voltammetry (CV) and ascertain changes in charge storage capacity (CSC). Testing was performed using an eDAQ potentiostat in combination with eChem software (eDAQ Pty Ltd., Australia). The linear voltage sweep was set to range within the limits of the water window, between −0.6 and 0.8V vs. an isolated Ag/AgCl reference with a large, low impedance Pt counter electrode. A sweep rate of 150 mV/s was applied for a total of 50 cycles, before CSC was calculated by integrating the current response with respect to time. Four electrodes of each surface type were constructed on each array and three arrays were used in total.

Electrochemical impedance spectroscopy (EIS) was performed on an eDAQ system coupled with the ZMan software (eDAQ Pty Ltd., Australia). Samples were analysed by application of a 50 mV sinusoid across a range of frequencies tested from 1 Hz to 100 kHz. Bode plots were produced of the impedance magnitude and phase response.

### In Vitro Electrochemical Charge Injection Limit

Many neuroprosthetic devices use current-controlled biphasic pulses to stimulate neural tissue. In the resulting potential transient produced from biphasic stimulation, the Vₐ (access voltage), is the instantaneous voltage change when a biphasic pulse is applied or removed. The E_{mc} (maximum cathodic voltage) is found at the termination of this voltage, where the transient potential begins to decay asymptotically towards the open circuit voltage in the absence of active electrode shorting or current reversal. The electrochemical limit used to define safe charge injection for cathodic first, biphasic current injection, is when E_{mc}, is at the reduction potential for water. This is nominally at −0.83 V, or more practically −0.61 V vs Ag/AgCl. In this study the charge injection limit was determined in two ways: firstly using a conventional isolated Ag/AgCl reference electrode, and then repeated using a Pt microelectrode which was part of the array and adjacent to the test electrode as the reference. This second technique was developed to enable in vivo assessment of electrochemical charge injection limits. Pt electrodes have been shown to be suitable as reference electrodes, demonstrating stability across the relevant temperature range with repeatable hysteresis. Since the reference electrode in this setup is also Pt, the voltage of the electrochemical limit is −0.83 V. The counter electrode in both arrangements was a large, low impedance Pt electrode placed at a distance more than 10 times that of the displacement between the reference and working electrode, to eliminate contamination of the reference recording.

These two reference electrode arrangements were compared in vitro to assess the accuracy of this technique for use in vivo. In vitro studies were performed in Dulbecco's phosphate buffered saline (DPBS) with a custom-built biphasic stimulator which delivered current-controlled charge-balanced biphasic pulses. The interphase delay was set at 0.01 ms and limits were assessed for phase durations ranging from 0.1-0.8 ms in 0.1 ms increments. The current was initially applied at 10 μA and was automatically increased in 1 μA increments using in-house software until E_{mc} reached the −0.61 V and −0.83 V for the Ag/AgCl and Pt references, respectively.

### In Vivo Electrochemical Charge Injection Limit

All experiments were conducted as a sub-project within a larger study with prior approval from the Animal Care and Ethics Committee at the University of New South Wales, and in accordance with the Australian Research Council guidelines for animal experimentation. Adult cats (n=3) were anesthetised and implanted with electrode arrays in the suprachoroidal space as detailed previously in Wong et al. Potential transients were recorded to confirm electrodes were functioning and in contact with the tissue. The in vivo charge injection limit was determined using adjacent electrodes on the array as the reference electrode, as specified above. A large, low impedance Pt counter electrode was placed sub-conjunctivally, within the orbit. Measurements were made in conjunction with other visual percept investigations, presented elsewhere, and hence these studies were performed at 36 hr post implantation.

### Electrode Stability

The stability of electrode surfaces was explored through electrical ageing with 150 million continuous stimulations applied vs. a monopolar return bathed in DPBS. A custom 24-channel stimulator was used to apply sequential 0.4 ms, 250 μA pulses. These parameters were determined in previous in vivo studies to elicit a cortical response in the feline visual cortex. The inter-phase delay was 20 μs and inter-stimulus delay was set to 2 ms. The arrays were maintained at 37° C. for 35 days. The voltage transient was captured on an isolated oscilloscope (Tektronix, USA). The DPBS was refreshed routinely to avoid any increase in ion concentration due to evaporation. SEM images were also taken to compare surface profiles following stimulation.

TABLE 1

| | A | B | C | D | E | F | G | H | I | J |
|---|---|---|---|---|---|---|---|---|---|---|
| 1 | pulse width | inter-stim | inter-phase | | mg/pulse | | pulses/second | | | |
| 2 | 0.4 | 0.02 | 2 | | 2.82 | | 354.509929 | | | |
| 3 | | | | | | | | | | |
| 4 | pulses/hour | | pulses/day | | pulses/35 days | | if divided by 24 | | | |
| 5 | 1276595.74 | | 30638297.9 | | 1.07E−09 | | 4.47E−07 | | | |
| 6 | | | | | | | | | | |

### Results

### Fabrication of Electrode Arrays

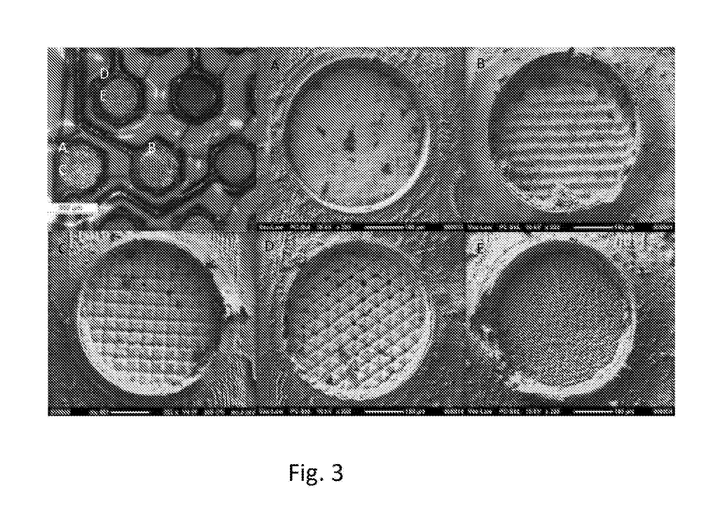

The electrodes were produced such that each surface type was represented on a single electrode array, with even distribution of each surface type across the array. This was designed such that there was no spatial bias when the electrode array was implanted which could lead to different surfaces being subjected to lower or higher tissue impedances related to geographical variability and other dynamics such as clotting and inflammation. The SEM images pictured in FIG. 3 clearly show the different structures which have been produced at the electrode surface. The Nd:YAG roughened electrode surface was the most difficult to produce as alignment between the two laser types was prone to errors which led to an off-centered pattern. As such this pattern was confined to the central region of the electrode, with some smooth areas in the border region and was expected to have a slightly reduced impact on electrode properties compared to previous studies.

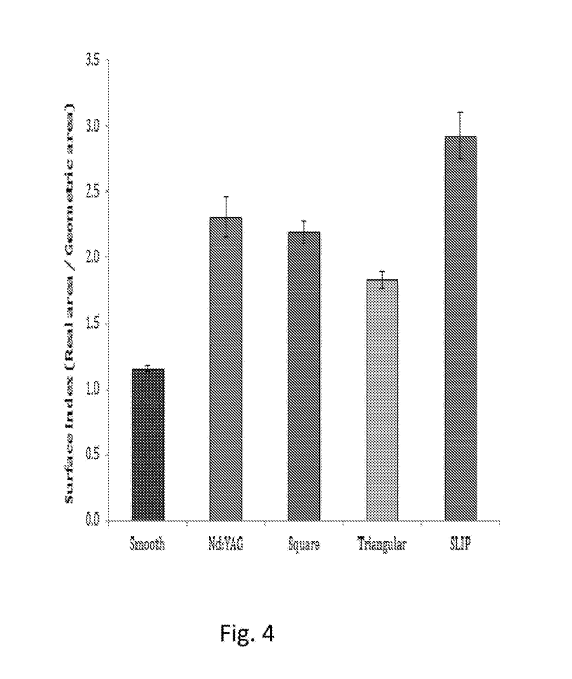

The optical profilometry measurements yielded a surface index (SI) which indicate the SLIP electrodes have the highest surface area with an SI of 2.9, see FIG. 4. For these electrodes with a nominal geometric area of 0.11 mm², the nanoroughened electrodes have a real surface area of 0.33 mm². Similarly the Nd:YAG roughened electrodes have a real surface area of 0.26 mm², the square electrodes are 0.25 mm² and the triangle electrodes are 0.21 mm². The smooth electrodes were also not perfectly flat, having a SI of 1.15 and a real surface area of 0.13 mm². FIG. 3 shows the representative surfaces for each laser pattern on the PT microelectrodes being A. smooth, B. Nd:YAG roughened, C. square, D. triangular and E. SLIP. FIG. 4 shows the increase in surface area imparted by laser roughening in graphical form.

### 3.2 Baseline Electrical Properties

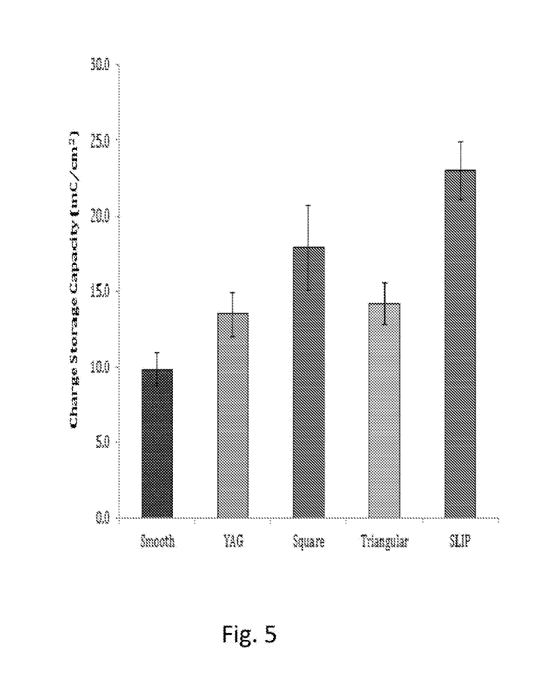

Cyclic voltammetry demonstrated that there was an increase in CSC, plotted in FIG. 5, related to the increased electrode surface area. This is shown by the change in CSC from 9.7 mC/cm² for the smooth surface to 13.5 mC/cm² for the Nd:YAG roughened electrodes, 14.1 mC/cm² for the triangular and 17.8 mC/cm² for the square patterned electrodes. The SLIP technique imparted the greatest increase with a total CSC of 23.0 mC/cm², being 2.3 times greater than the smooth electrodes.

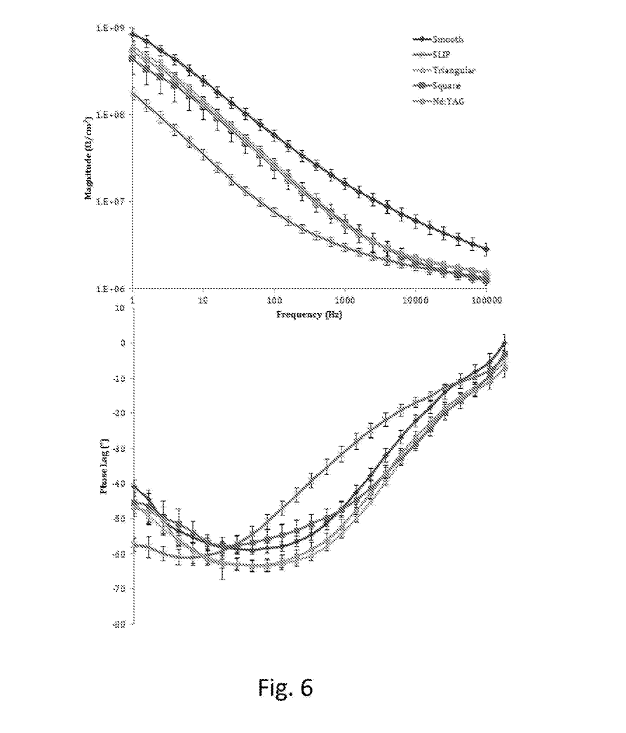

EIS was performed to determine the opposition of a surface to the flow of charge. The impedance response is shown as a Bode plot in FIG. 6. As expected, at all frequencies modified surfaces imparted a decrease in impedance magnitude. Even at high frequency (10-100 kHz) the impedance magnitude of the smooth surface is almost double that of the SLIP surface. The SLIP surface also presents with a distinct phase shift, which reduces phase lag in comparison to smooth electrodes at frequencies greater than 10 Hz. The Nd:YAG roughened, square and triangular structured electrodes present with very similar impedance responses which reflect their similarity in surface area.

### 3.4 Charge Injection Limits

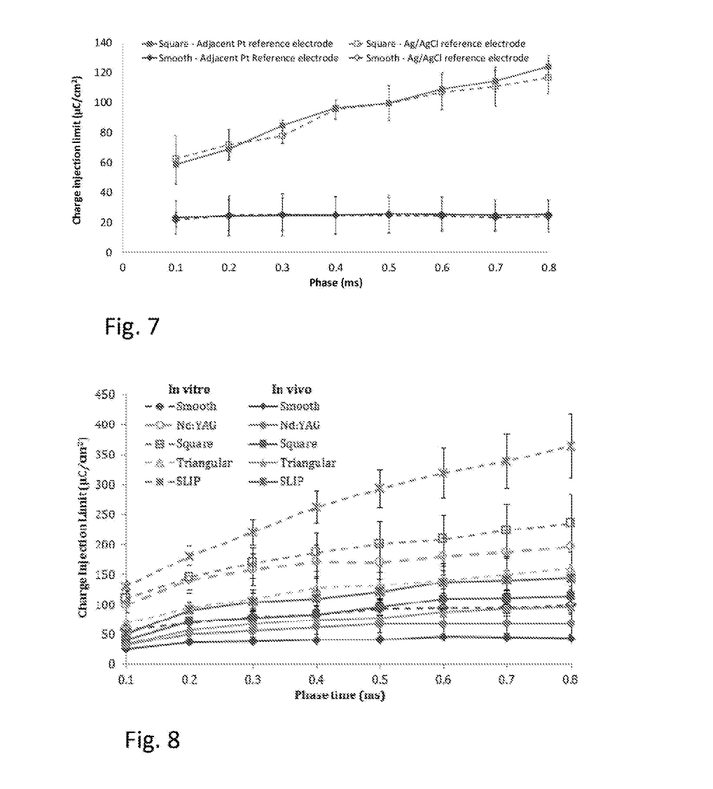

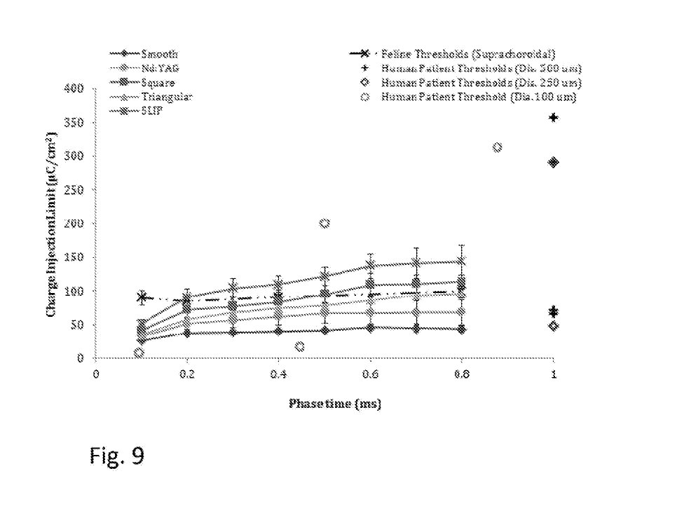

The electrochemical charge injection limit is rarely assessed in the implant environment due to the limitations in forming a 3-electrode cell which can adequately assess voltage transients on microelectrode arrays. In this study validation was obtained of a 3-electrode set up which makes use of the adjacent electrodes on the array as reference electrodes, to facilitate assessment of in vivo injection limits. The comparison of the two methods is presented in FIG. 7 for two types of electrode surfaces, being the conventional smooth electrodes and the square patterned electrodes. It can be easily seen that the two methods are comparable with the maximum deviation between the two metrics being 9% and the average deviation being 4%, with no statistically significant difference across the phase duration spectrum.

For simplicity, in vitro electrode injection limits for all arrays are shown in comparison to the in vivo values in FIG. 8. The different electrode surfaces were shown to produce electrochemical injection limits which concurred with other electrochemical metrics. As expected all surfaces presented a phase dependent result with the injection limit increasing with the phase duration. The SLIP surface produced injection limits in saline which ranged from 130-364 μC/cm² across the 0.1-0.8 ms phase range, compared to the smooth electrodes which varied from 58-98 μC/cm². The Nd:YAG roughened and square patterned arrays had very similar results of 100-197 μC/cm² and 119-235 μC/cm², respectively. The triangular pattern had lower limits ranging from 69-160 μC/cm² across the same phase durations.

### 3.5 In Vivo Electrode Characterisation

In the feline model in vivo electrochemical charge injection limits were obtained through utilizing adjacent electrodes within the array as reference electrodes. The in vivo charge injection was compared to values obtained in physiological saline in FIG. 8. The relative relationships of electrode performance across the range of phase durations was consistent with the in vitro data, but with a significant reduction in total charge which can be transduced before reaching the electrochemical potential for water reduction. The percentage reduction in injection limit from in vitro to in vivo presented in Table 1, was consistent across all phase durations. The Nd:YAG roughened surface experienced the highest drop in charge injection limit of 63.8±1.9%, yielding a limit range of 33-68 μC/cm² followed by the SLIP surface with 57.1±3.2% or 51-144 μC/cm². Surprisingly, the triangular patterned surface experienced the least reduction with a loss of only 40.4±3.3% (35-96 μC/cm²), which is 12% better than smooth electrodes with a charge injection limit of 26-44 μC/cm² across the phase range of 0.1-0.8 ms.

TABLE 2
Average drop in charge injection (CI) limit
from in vitro to in vivo, (n = 12).

| Surface morphology | % drop in CI limit | SD |
|---|---|---|
| Smooth | 52.0 | ±3.2 |
| Nd:YAG | 63.8 | ±1.9 |
| Square | 53.1 | ±3.4 |
| Triangular | 40.4 | ±3.3 |
| SLIP | 57.1 | ±3.2 |

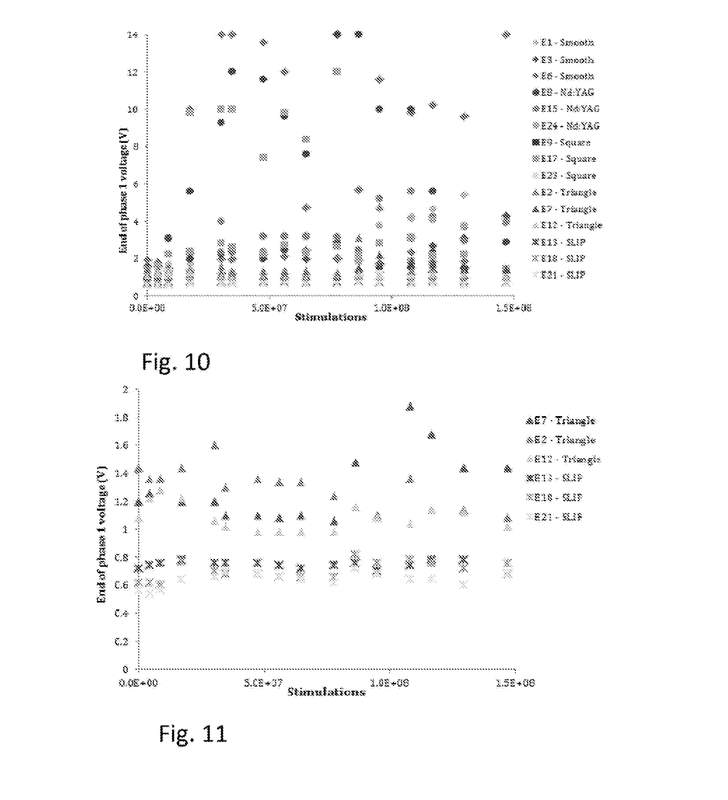

The in vivo injection limit results are plotted with respect to visual percept thresholds obtained from retinal stimulation in both feline model with suprachoroidal device placement and human patients with epiretinal or subretinal device placement in FIG. 10 alongside threshold values which have been reported in the literature to elicit visual percepts in both animal models and human patients. The diameter of electrodes used in the various human trials is also noted. In the feline model it is clear that the smooth electrodes experience electrochemical limits at values lower than the threshold for visual cortex activation (˜90 μC/cm²), but the SLIP and square patterned electrodes have injection limits which supersede this value at longer phase durations. The SLIP electrodes have an electrochemical injection limit higher than threshold at 0.2 ms and above, where the square electrodes have an injection limit above threshold from 0.5 ms onwards. Of ten patient visual percept thresholds reported in literature, only two are within the electrochemical limits of smooth Pt electrodes.

### 3.3 Electrode Stability

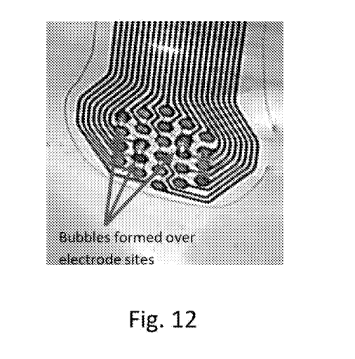

All electrodes were subjected to 150 million stimulations at clinically relevant levels, and the potential transient at the end of phase 1 was plotted in FIG. 11 for one array with three electrodes of each surface type. Results are shown for one array, but the experiment was repeated across three arrays with repeatable results. The results shown are for end of phase one voltage on electrodes under continuous biphasic stimulation for 150 million pulses, n=3. The variation in the voltage is an indirect measure of the stability of the electrode impedance. The SLIP electrodes consistently experienced the lowest end of phase 1 voltage, ranging from 0.6-0.82 V. FIG. 12 clearly shows the stability of the SLIP electrodes across the study period. While there was more variability on the triangular electrodes, the voltage was consistently below 2.0 V. The square patterned, Nd:YAG roughened and smooth electrodes all experienced considerable variability in voltage transient across the study period, with the end of phase voltage ranging from 1.0-14.0 V. In all of these electrode types the end of phase 1 voltage more than doubled from the starting voltage at repeated time points throughout the study. It was observed that gassing occurred on these electrode types, resulting in the formation of bubbles (see FIG. 13), which corresponded with open circuit voltage recordings (14 V device compliance). It was however, noted that the bubble formation also protected the electrode from dissolution, and agitation of the electrolyte to remove the bubbles returned the voltage transient to a normal magnitude which was typical for each surface type.

The SEM images support the potential transient data, showing that SLIP and triangular-patterned electrodes were stable under continuous stimulation. The square electrodes which experienced large increases in voltage were found to have significant surface dissolution. The smooth electrodes had some evidence of surface pitting, but qualitative observations do not show definitive dissolution. It is important to note that electrodes on which bubbles formed presented as an open circuit and were inherently protected by that bubble from further damage by dissolution. Ultimately, these open circuit electrodes were subjected to fewer stimuli than the electrodes on which bubbles did not form. Repeat studies on three electrode arrays were performed in a stirred medium to simulate shear forces, but recurrent bubble formation continued to hamper attempts to elicit thresholds for electrode dissolution. All arrays (a total of 12 electrodes of each surface type) experienced similar results across the studies with gassing and doubling of the voltage experienced by 100% of smooth electrodes, 11% of triangular, 55% of square, 67% of Nd:YAG electrodes under stimulation conditions. Neither gassing nor significant increases in end of phase voltage were experienced by the SLIP electrodes.

Improvements in charge transfer were shown through electrochemical impedance spectroscopy (EIS), cyclic voltammetry (CV) and biphasic stimulation at clinically relevant levels. A new method was investigated and validated which enabled the assessment of in vivo electrochemically safe charge injection limits. All of the modified surfaces provided electrical advantage over the smooth Pt. The SLIP surface provided the greatest benefit both in vitro and in vivo, and this surface was the only type which had injection limits above the threshold for neural stimulation, at a level shown to produce a response in the feline visual cortex when using an electrode array implanted in the suprachoriodal space of the eye. This surface was found to be stable when stimulated with 1 billion clinically relevant pulses.

Critical to the assessment of implant devices is accurate determination of safe usage limits in an in vivo environment. Laser patterning, in particular SLIP, is a superior technique for improving the performance of implant electrodes without altering the interfacial electrode chemistry through coating.

While the method has been described in reference to its preferred embodiments, it is to be understood that the words which have been used are words of description rather than limitation and that changes may be made without departing from the scope of the application as defined by the appended claims.

It is to be understood that a reference herein to a prior art document does not constitute an admission that the document forms part of the common general knowledge in the art in Australia or in any other country.

In the claims which follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the method of the disclosure.

1. A method of treating a metallic surface comprising:
exposing the surface to laser pulses at an energy density below the threshold for ablation of bulk material for the metallic surface; maintaining the exposure until a multiplicity of pores form in the surface.

2. A method of treating a surface as defined in claim 1, wherein the metallic surface is the surface of a platinum electrode.

3. A method of treating a surface as defined in claim 1, wherein the exposure is maintained through a rippled surface effect until a porous surface structure is produced.

4. A method of treating a surface as defined in claim 1, wherein the step of exposing the surface to laser pulses is performed greater than 1000 times.

5. A method of treating a surface as defined in claim 1, wherein the surface is exposed to greater than 20000 laser pulses.

6. A method of enhancing the electrochemical surface area of an electrode comprising treating the surface of the electrode by applying laser pulses at an energy density below the threshold for ablation of bulk material for the surface of the electrode and repeating the exposure until a porous surface is formed on the electrode.

7. A method of enhancing the electrochemical surface area of an electrode as defined in claim 6, wherein the exposure is maintained through a rippled surface effect until a porous surface structure is produced on the electrode.

8. A method of enhancing the electrochemical surface area of an electrode as defined in claim 6, wherein the exposure is maintained through a rippled surface effect until a porous surface structure is produced.

9. A method of enhancing the electrochemical surface area of an electrode as defined in claim 6, wherein the step of exposing the surface to laser pulses is performed greater than 1000 times.

10. A method of enhancing the electrochemical surface area of an electrode as defined in claim 6, wherein the surface is exposed to greater than 20000 laser pulses.

11. A method of forming an electrode for use within a medical device, the method comprising:
treating a surface of a platinum electrode by exposing the surface to laser pulses at an energy density below the threshold for ablation of bulk platinum;
maintaining the exposure until a multiplicity of pores form in the surface.

12. A method of forming an electrode for use within a medical device as defined in claim 11, wherein the medical device is a visual prosthesis, a cochlea implant, a peripheral nerve implant or a deep brain stimulator.

13. A method of forming an electrode for use within a medical device as defined in claim 11, wherein the medical device is a retinal implant.
